(19) United States
(12) Patent Application Publication
(10) Pub. No.: US 2024/0424098 A1
(43) Pub. Date: Dec. 26, 2024

(54) MODIFIED IMMUNE CELLS AND METHODS OF USE THEREOF

(71) Applicant: UNIVERSITY HEALTH NETWORK

(72) Inventors: Daniel Diniz DE CARVALHO, Toronto (CA); Helen LOO YAU, Toronto (CA); Ilias ETTAYEBI, Burlington (CA)

(73) Assignee: UNIVERSITY HEALTH NETWORK, Toronto (CA)

(21) Appl. No.: 18/699,121

(22) PCT Filed: Oct. 5, 2022
(86) PCT No.: PCT/IB2022/059520

§ 371 Date: Apr. 5, 2024

Related U.S. Application Data
(60) Provisional application No. 63/253,001, filed on Oct. 6, 2021.

(51) Int. Cl. A61K 39/00; A61P 35/00; C12N 5/0783; C12N 9/22; C12N 15/11; C12N 15/90

(52) U.S. Cl. CPC A61K 39/4611; A61K 39/4631; A61K 39/4632; A61K 39/464488; A61P 35/00; C12N 5/0636; C12N 9/22; C12N 15/11; C12N 15/907; C12N 2310/20; C12N 2510/00

(57) ABSTRACT

The present disclosure is directed to methods of treating a subject in need thereof, comprising administering to the subject a population of modified immune cells, which comprises one or more modified immune cells having decreased expression of one or more components of the SAGA (Spt-Ada-Gen5-acetyltransferase) complex, relative to an unmodified immune cell. In some aspects, the immune cell comprises a chimeric antigen receptor or a T cell receptor. In some aspects. the immune cell is a T cell, an NK cell, or a tumor infiltrating lymphocyte (TIL).

### CROSS-REFERENCE TO RELATED APPLICATIONS

This application claims priority benefit of U.S. Provisional Application No. 63/253,001, filed Oct. 6, 2021, which is herein incorporated by reference in its entirety.

### FIELD OF THE DISCLOSURE

The present disclosure provides methods for treating a disease or condition in a subject comprising administering modified immune cells, wherein the modified immune cells comprise depleted SAGA complex.

### BACKGROUND OF THE DISCLOSURE

Immunotherapy has emerged as a critical tool in the battle against a variety of diseases, including cancer. T cell therapies, including chimeric antigen receptor (CAR) T cell therapies and engineered T cell receptor (TCR) cell therapies, are at the forefront of immunotherapeutic development. Adoptive transfer of antitumor T cells has been shown to induce clinical responses in cancer patients.

However, T cell therapies are limited by the ability of transplanted T cells to maintain anticancer activity and potency in vivo. Transplanted T cells often lose efficacy rapidly in vivo, as T cells become terminally differentiated and exhausted. Various means of reducing or blocking T cell exhaustion and increasing potency are being explored, however, there remains a need in the field of cancer immunotherapy to increase the persistence of adoptively transferred T cells, including CAR-T and engineered TCR T cells, thereby increasing in vivo efficacy. T cell therapies can also be limited by their potency to kill target cancer cells.

### SUMMARY OF THE DISCLOSURE

Certain aspects of the present disclosure are directed to a method of treating a subject in need thereof, comprising administering to the subject a population of modified immune cells, which comprises one or more modified immune cells having decreased expression of one or more components of the SAGA (Spt-Ada-Gcn5-acetyltransferase) complex, relative to an unmodified immune cell. In some aspects, the population of modified immune cells are modified by culturing the immune cells ex vivo under conditions that block or reduce the expression of one or more components of the SAGA complex prior to administration to the subject.

Some aspects of the present disclosure are directed to a method of treating a subject in need thereof, comprising (i) culturing a population of immune cells ex vivo under conditions that block or reduce the expression of one or more components of the SAGA thereby generating a population of modified immune cells, and (ii) administering the population of modified immune cells to the subject.

In some aspects, the population of immune cells comprises one or more immune cells selected from the group consisting of T cells, TsCM cells, double negative T (DNT) cells, natural killer (NK) cells, B cells, regulatory T (Treg) cells, tumor infiltrating lymphocytes, and any combination thereof. In some aspects, the population of immune cells comprises CD8+ T cells.

In some aspects, the modified immune cell comprises a nucleic acid encoding a chimeric antigen receptor (CAR) or a nucleic acid encoding a heterologous T cell receptor (TCR). In some aspects, the CAR, the TCR, or both comprise an antigen-binding domain, wherein the antigen-binding domain specifically binds an antigen expressed on the surface of a tumor cell. In some aspects, the antigen-binding domain specifically binds an antigen selected from the group consisting of CD19, BCMA, CD30, CD33, CD123, FLT3, and any combination thereof. In some aspects, the antigen-binding domain specifically binds an antigen selected from the group consisting of NYESO-1, MAGE-A1, MAGE-A2, MAGE-A3, MAGE-A4, MAGE-A6, MAGE-A8, MAGE-A9, MAGE-A10, MAGE-A12, MART-1, gp100, WTI, tyrosinase, PRAME, p53, HPV-E6, HBV, TRAIL&DR4, thyroglobulin, TGFBII frameshift antigen, LAGE-1A, KRAS G12V, HPV-E7, HERV-E, HA-1, CMV, CEA, AFP, and any combination thereof.

In some aspects, the one or more components of the SAGA complex comprise USP22, TADA1, TADA2b, TAF6L, or any combination thereof. In some aspects, the expression of the one or more components of the SAGA complex is inhibited or blocked by contacting the immune cells with (i) a Cas9 protein and a CRISPR-cas system guide RNA (gRNA), (ii) an antisense oligonucleotide, (iii) an siRNA, (iv) an shRNA, (v) a small molecule inhibitor, (vi) a prime editing guide RNA, or (vii) any combination thereof. In some aspects, the expression of the one or more components of the SAGA complex is inhibited or blocked by contacting the immune cells with a Cas9 protein and a CRISPR-cas system guide RNA (gRNA). In some aspects, the Cas9protein, the gRNA, or both are expressed from a heterologous expression construct introduced into the immune cell. In some aspects, the gRNA is a single guide RNA (sgRNA).

In some aspects, the gRNA specifically hybridizes to a target DNA region within a coding region encoding a component of the SAGA complex selected from the group consisting of USP22, TADA1, TADA2b, and TAF6L. In some aspects, the gRNA specifically hybridizes to a target DNA region within the USP22 coding region. In some aspects, the gRNA specifically hybridizes to a target DNA region within the TADAI coding region. In some aspects, the gRNA specifically hybridizes to a target DNA region within the TADA2b coding region. In some aspects, the gRNA specifically hybridizes to a target DNA region within the TAF6L coding region.

In some aspects, the method comprises contacting the immune cell with a first gRNA and a second gRNA, wherein the first gRNA hybridizes to a target DNA region within a coding region encoding a first component of the SAGA complex, wherein the second gRNA hybridizes to a target DNA region within a coding region encoding a second component of the SAGA complex, and wherein the first component of the SAGA complex and the second component of the SAGA complex are different. In some aspects, (i) the first component of the SAGA complex is USP22, and the second component of the SAGA complex is TADA1; (ii) the first component of the SAGA complex is USP22, and the second component of the SAGA complex is TADA2b; (iii) the first component of the SAGA complex is USP22, and the second component of the SAGA complex is TAF6L; (iv) the first component of the SAGA complex is TADA1, and the second component of the SAGA complex is TADA2b; (v) the first component of the SAGA complex is TADA1, and the second component of the SAGA complex is TAF6L; or (vi) the first component of the SAGA complex is TADA2b, and the second component of the SAGA complex is TAF6L.

In some aspects, the method further comprises contacting the immune cell with a third gRNA, wherein the third gRNA hybridizes to a target DNA region within a coding region encoding a third component of the SAGA complex; and wherein the first component of the SAGA complex, the second component of the SAGA complex, and the third component of the SAGA complex are different. In some aspects, (i) the first component of the SAGA complex is USP22, the second component of the SAGA complex is TADAI, and the third component of the SAGA complex is TADA2b; (ii) the first component of the SAGA complex is USP22, the second component of the SAGA complex is TADA1, and the third component of the SAGA complex is TAF6L; (iii) the first component of the SAGA complex is TADA1, the second component of the SAGA complex is TADA2b, and the third component of the SAGA complex is TAF6L; or (iv) the first component of the SAGA complex is USP22, the second component of the SAGA complex is TADA2b, and the third component of the SAGA complex is TAF6L.

In some aspects, the method further comprises contacting the immune cell with a fourth gRNA, wherein the fourth gRNA hybridizes to a target DNA region within a coding region encoding a fourth component of the SAGA complex; and wherein the first component of the SAGA complex, the second component of the SAGA complex, the third component of the SAGA complex are different, and the fourth component of the SAGA complex are different. In some aspects, the first component of the SAGA complex is USP22, the second component of the SAGA complex is TADA1, the third component of the SAGA complex is TADA2b, and the fourth component of the SAGA complex is TAF6L. In some aspects, contacting the immune cells with the Cas9 protein and the gRNA knocks out the target component of the SAGA complex in the modified immune cell.

In some aspects, the modified immune cells exhibit increased persistence, relative to unmodified immune cells.

In some aspects, the modified immune cells exhibit increased expression of one or more activation markers selected from the group consisting of CD25, HLA-DR, CD69, and any combination thereof following re-stimulation, relative to unmodified immune cells following re-stimulation. In some aspects, the re-stimulation comprises contacting the cells with CD3, CD28, CD2, or any combination thereof. In some aspects, the re-stimulation is applied at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after an initial stimulation.

In some aspects, the modified immune cells exhibit increased expression of one or more activation markers selected from the group consisting of CD25, HLA-DR, CD69, and any combination thereof following serial stimulation, relative to unmodified immune cells following serial stimulation. In some aspects, the serial stimulation comprises a first stimulation, a first re-stimulation, and a second re-stimulation. In some aspects, the serial stimulation further comprises a third re-stimulation. In some aspects, the serial stimulation further comprises a fourth re-stimulation.

In some aspects, the modified immune cells exhibit increased expression of one or more activation markers after serial stimulation, wherein the one or more activation markers are selected from the group consisting of CD25, HLA-DR, CD69, and any combination thereof at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, or at least about 41 days after an initial stimulation, relative to unmodified immune cells following serial stimulation. In some aspects, the expression of the one or more activation markers is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to unmodified immune cells following re-stimulation.

In some aspects, the modified immune cells exhibit increased expression of CD107a following re-stimulation, relative to unmodified cells following re-stimulation. In some aspects, the expression of CD107a is increased by at least about 10%, at least about 15%, at least about 20%, at least about 25%, at least about 30%, at least about 40%, at least about 50%, at least about 75%, or by at least about 100%, relative to unmodified cells following re-stimulation.

In some aspects, the modified immune cells exhibit decreased expression of one or more T cell exhaustion markers following re-stimulation, relative to unmodified immune cells. In some aspects, the one or more T cell exhaustion markers are selected from the group consisting of LAG3 and TIM3. In some aspects, the expression of the one or more T cell exhaustion markers is decreased by at least about 10%, at least about 15%, at least about 20%, at least about 25%, at least about 30%, at least about 40%, at least about 50%, or at least about 75%, relative to unmodified cells following re-stimulation.

In some aspects, the subject is afflicted with a cancer. In some aspects, the cancer is selected from the group consisting of melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of the cancers. In some aspects, the cancer is relapsed, refractory, advanced, and/or metastatic.

In some aspects, the method further comprises administering to the subject an additional anticancer agent. In some aspects, the additional anticancer agent comprises an immunotherapy, a chemotherapy, a cytokine, a radiation therapy, a surgery, or any combination thereof. In some aspects, the additional anticancer agent comprises a cell based immunotherapy, an antibody or an antigen-binding portion thereof, or both. In some aspects, the additional anticancer agent comprises a checkpoint inhibitor. In some aspects, the additional anticancer agent comprises a PD-1 inhibitor, a PD-L1 inhibitor, a CTLA-4 inhibitor, a LAG3 inhibitor, a TIM3 inhibitor, or any combination thereof. In some aspects, the additional anticancer agent comprises an antibody or an antigen-binding portion thereof that specifically binds PD-1 or PD-L1.

In some aspects, the modified immune cells exhibit increased phospho-MEK levels following stimulation, relative to unmodified cells following stimulation. In some aspects, the modified immune cells exhibit increased phospho-ATK levels following stimulation, relative to unmodified cells following stimulation.

### BRIEF DESCRIPTION OF THE DRAWINGS

FIG. 1A is a schematic representation of a timeline for SAGA target validation to measure CD25, CD69, and HLA-DR on CD8+ T cells at Day 14 and Day 28. FIGS. 1B-1M are flow cytometry plots of extracellular expression of activation markers CD25 (FIGS. 1B-1E), HLA-DR (FIGS. 1F-1I), and CD69 (FIGS. 1J-1M) in CD8+ T cells at resting (FIGS. 1B, 1F, and 1J), at 24-hour re-stimulation (FIGS. 1C, 1G, and 1K), at day-21 re-stimulation (FIGS. 1D, 1H, and 1L), and day-28 re-stimulation (FIGS. 1E, 1I, and 1M), as outlined in FIG. 1A (N=1 biological sample). FIGS. 1N-1S are graphical representations of the fold change in expression of activation markers CD25 (FIGS. 1N and 1Q), CD69 (FIGS. 1O and 1R), and HLA-DR (FIGS. 1P and 1S) in CD8+ T cells depleted of SAGA-complex components USP22, TADA1, TADA2B, TAF6L, TRRAP, and KAT2a, as indicated (x-axes) relative to control (CTL) measured at Days 14 and 28 post re-stimulation. Fold change expression is normalized to resting control conditions (N=1 biological sample). FIGS. 1T-1Y are scatter plots of CD107a percentage level expression in CD8+ T cells depleted of SAGA-complex components USP22 (FIG. 1U), TADAI (FIG. 1V), TADA2b (FIG. 1W), TAF6L (FIG. 1X), and TRRAP (FIG. 1Y) relative to gControl (gC_{TL}; FIG. 1T) measured at Day 28 in vitro (N=1 biological sample).

FIG. 2A is a schematic representation of a timeline for SAGA target validation to measure activation markers CD25, CD69, and HLA-DR on CD8+ T cells at Day 14 and Day 41 and exhaustion markers LAG3 and TIM3 at Day 41. FIGS. 2B-2I are graphical representations of Fold change expression of activation markers CD25 (FIGS. 2B-2C), HLA-DR (FIGS. 2D-2E), and CD69 (FIGS. 2F-2G) and exhaustion markers LAG3 (FIG. 2H) and TIM3 (FIG. 2I) in CD8+ T cells depleted of SAGA-complex components USP22, TADA1, TADA2B, TAF6L, TRRAP, and KAT2a, as indicated (x-axes) relative to control (CTL) measured at Days 14 and 41 (FIGS. 2B-2G) or Day 41 (FIGS. 2H-2I) post re-stimulation. Fold change expression is normalized to resting control conditions (N=1 biological sample).

FIG. 3A is a line graph illustrating validation of target killing potential by antigen specific (NYESO-1) CD8 T cells multiplexed with SAGA target knock out (TADAI KO, TADA2B KO, TAF6L KO, or USP22 KO, as indicated) as measured by luciferase luminescence (N=1 biological sample). FIGS. 3B-3D are scatter plots of CD107a percentage level expression in transgenic TCR (NYESO-1 specific) CD8+ T cells multiplexed with SAGA target knock outs USP22 (FIG. 3C), TADAI (FIG. 3D), TADA2b (FIG. 3E), and TAF6L (FIG. 3F) relative to gControl (gC_{TL}; FIG. 3B) (N=1 biological sample).

FIGS. 4A-4C are graphical representations of the results of an ATAC-seq analysis showing transcription factor motifs with gained accessibility (FIG. 4A) and the levels of phospho-MEK (FIG. 4B) and phosphor-AKT (FIG. 4C) for gTADAI KO, gTADA2b KO, and gTAF6L KO relative to gControl.

FIG. 5A is a schematic representation of a co-culture assay to assess the in vitro killing of A375 melanoma cancer cells by transgenic NYESO-1+ T cells depleted of SAGA targets: gTADAI KO, gTADA2b KO, gTAF6L KO versus gC_{TL}. FIGS. 5B-5D are graphical representations of target cell killing as measured by A375 recovery at E:T ratios of 1:4 (FIG. 5B), 1:2 (FIG. 5C), and 1:1 (FIG. 5D).

FIGS. 6A-6D show the results of in vivo effects of SAGA-depleted NY-ESO-1 TCR+ T cells administered to NSG mice, engrafted with A375 melanoma cells. FIGS. 6A and 6B show tumor volume at 9 days and 15 days post adoptive cell transfer (ACT), respectively. FIGS. 6C and 6D show tumor images (FIG. 6C) and measured tumor weights (FIG. 6D) at day 17 post ACT.

### DETAILED DESCRIPTION OF THE DISCLOSURE

The present disclosure is directed to methods of treating a subject in need thereof, comprising administering to the subject a population of modified immune cells, which comprises one or more modified immune cells having decreased expression of one or more components of the SAGA (Spt-Ada-Gcn5-acetyltransferase) complex, relative to an unmodified immune cell. In some aspects, the population of immune cells are modified by culturing the immune cells ex vivo under conditions that block or reduce the expression of one or more components of the SAGA complex prior to administration to the subject. Some aspects of the present disclosure are directed to methods of treating a subject in need thereof, comprising (i) culturing a population of immune cells ex vivo under conditions that block or reduce the expression of one or more components of the SAGA thereby generating a population of modified immune cells, and (ii) administering the population of modified immune cells to the subject.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell).

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject,” as used herein, refers to a human. The terms “subject” and “patient” are used interchangeably herein. As used herein, the phrase “subject in need thereof” includes human subjects that would benefit, e.g., from administration of a composition comprising a modified immune cell of the disclosure, e.g., to control tumor growth.

The term “therapeutically effective amount” or “therapeutically effective dosage” refers to an amount of an agent (e.g., a modified immune cell of the disclosure) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., a modified immune cell disclosed herein) can, for example, (i) reduce the number of cancer cells; (ii) reduce tumor size; (iii) inhibit, reduce, slow to some extent and can stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., a modified immune cell disclosed herein), which is clinically shown to effect a significant decrease in cancer or slowing of progression (regression) of cancer, such as an advanced solid tumor. The ability of a therapeutic agent of the present disclosure (e.g., a modified immune cell disclosed herein) to promote disease regression can be evaluated using a variety of methods, such as in human subjects during clinical trials, in animal model systems predictive of efficacy in humans, or by assaying the activity of the agent in in vitro assays.

By way of example, an “anticancer agent” promotes cancer regression in a subject or prevents further tumor growth. In certain aspects, a therapeutically effective amount of the anticancer agent (e.g., a modified immune cell disclosed herein) promotes cancer regression to the point of eliminating the cancer.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12 (4): 252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “chimeric antigen receptor” and “CAR,” as used herein, refer to a recombinant fusion protein that has an antigen-specific extracellular domain coupled to an intracellular domain that directs the cell to perform a specialized function upon binding of an antigen to the extracellular domain.

The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen binding agents by their ability to both bind MHC-independent antigen and transduce activation signals via their intracellular domain.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 μM, for example, about 0.1 pM to about 1 μM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains (cAb VHH (camelid antibody variable domains) and humanized versions thereof, IgNAR VH (shark antibody variable domains) and humanized versions thereof, sdAb VH (single domain antibody variable domains) and “camelized” antibody variable domains are suitable for use. In some aspects, T cell receptor (TCR) based recognition domains, such as single chain TCR (scTv, single chain two-domain TCR containing V.alpha. V.beta.) are also suitable for use.

A chimeric antigen receptor disclosed herein (e.g., comprising a chimeric polypeptide) can also include an intracellular domain that provides an intracellular signal to the cell (expressing the CAR) upon antigen binding to the antigen-specific extracellular domain. In some aspects, the intracellular signaling domain of a CAR is responsible for activation of at least one of the effector functions of the T cell in which the chimeric receptor is expressed.

The term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, 829, Fc RIII, Fc RI, and combinations of signaling molecules, such as CD3 zeta, and CD28, CD27, 4-1BB, DAP-10, OX40, and combinations thereof, as well as other similar molecules and fragments. Intracellular signaling portions of other members of the families of activating proteins can be used, such as FcγRIII and FcεRI. In some aspects, the entire intracellular domain is included in the CAR. In other aspects, the CAR comprises a portion of an intracellular domain disclosed herein. In some aspects, the antigen-specific extracellular domain is linked to the intracellular domain of the chimeric antigen receptor by a transmembrane domain. A transmembrane domain traverses the cell membrane, anchors the CAR to the T cell surface, and connects the extracellular domain to the intracellular signaling domain, thus impacting expression of the CAR on the T cell surface. Chimeric antigen receptors can also further comprise one or more costimulatory domain and/or one or more spacer. A costimulatory domain is derived from the intracellular signaling domains of costimulatory proteins that enhance cytokine production, proliferation, cytotoxicity, and/or persistence in vivo.

In some aspects, a “peptide hinge” or “spacer” connects the antigen-specific extracellular domain to the transmembrane domain. In some aspects, the transmembrane domain is fused to the costimulatory domain, optionally a costimulatory domain is fused to a second costimulatory domain, and the costimulatory domain is fused to a signaling domain, not limited to CD3ζ. For example, inclusion of a spacer domain between the antigen-specific extracellular domain and the transmembrane domain, and between multiple scFvs in the case of tandem CAR, can affect flexibility of the antigen-binding domain(s) and thereby CAR function. Suitable transmembrane domains, costimulatory domains, and spacers are known in the art.

The term “T cell receptor” (TCR), as used herein, refers to a heteromeric cell-surface receptor capable of specifically interacting with a target antigen. As used herein, “TCR” includes but is not limited to naturally occurring and non-naturally occurring TCRs; full-length TCRs and antigen binding portions thereof; chimeric TCRs; TCR fusion constructs; and synthetic TCRs. In human, TCRs are expressed on the surface of T cells, and they are responsible for T cell recognition and targeting of antigen presenting cells. Antigen presenting cells (APCs) display fragments of foreign proteins (antigens) complexed with the major histocompatibility complex (MHC; also referred to herein as complexed with an HLA molecule, e.g., an HLA class II molecule). A TCR recognizes and binds to the peptide: HLA complex and recruits CD8 (for MHC Class I molecules) or CD4 (for MHC class II molecules), activating the TCR. The activated TCR initiates downstream signaling and an immune response, including the destruction of the EPC.

In general, a TCR can comprise two chains, an alpha chain and a beta chain (or less commonly a gamma chain and a delta chain), interconnected by disulfide bonds. Each chain comprises a variable domain (alpha chain variable domain and beta chain variable domain) and a constant region (alpha chain constant region and beta chain constant region). The variable domain is located distal to the cell membrane, and the variable domain interacts with an antigen. The constant region is located proximal to the cell membrane. A TCR can further comprises a transmembrane region and a short cytoplasmic tail. As used herein, the term “constant region” encompasses the transmembrane region and the cytoplasmic tail, when present, as well as the traditional “constant region.”

The variable domains (of a CAR antigen binding domain or a TCR) can be further subdivided into regions of hypervariability, termed complementarity determining regions (CDRs), interspersed with regions that are more conserved, termed framework regions (FR). Each alpha chain variable domain and beta chain variable domain comprises three CDRs and four FRs: FR1, CDR1, FR2, CDR2, FR3, CDR3, FR4. Each variable domain contains a binding domain that interacts with an antigen. Though all three CDRs on each chain are involved in antigen binding, CDR3 is believed to be the primary antigen binding region, while CDR1 and CDR2 are believed to primarily recognize the HLA molecule.

Where not expressly stated, and unless the context indicates otherwise, the term “TCR” also includes an antigen-binding fragment or an antigen-binding portion of any TCR disclosed herein, and includes a monovalent and a divalent fragment or portion, and a single chain TCR. The term “TCR” is not limited to naturally occurring TCRs bound to the surface of a T cell. As used herein, the term “TCR” further refers to a TCR described herein that is expressed on the surface of a cell other than a T cell (e.g., a cell that naturally expresses or that is modified to express CD4, as described herein), or a TCR described herein that is free from a cell membrane (e.g., an isolated TCR or a soluble TCR).

An “antigen binding molecule,” “portion of a” CAR or TCR, or “fragment” refers to any portion of an CAR or TCR less than the whole. An antigen binding molecule can include the antigenic CDRs.

The term “autologous” refers to any material derived from the same individual to which it is later to be re-introduced. For example, an autologous T cell therapy comprises administering to a subject a T cell that was isolated from the same subject. The term “allogeneic” refers to any material derived from one individual which is then introduced to another individual of the same species. For example, an allogeneic T cell transplantation comprises administering to a subject a T cell that was obtained from a donor other than the subject.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present invention include, but are not limited to, cancers of the immune system including lymphoma, leukemia, and other leukocyte malignancies. In some aspects, the methods of the present invention can be used to reduce the tumor size of a tumor derived from, for example, bone cancer, renal cancer, prostate cancer, breast cancer, colon cancer, lung cancer, cutaneous or intraocular malignant melanoma, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. The particular cancer can be responsive to chemo- or radiation therapy or the cancer can be refractory.

A refractory cancer refers to a cancer that is not amendable to surgical intervention, and the cancer is either initially unresponsive to chemo- or radiation therapy or the cancer becomes unresponsive over time.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term “progression-free survival,” which can be abbreviated as PFS, as used herein refers to the time from the treatment date to the date of disease progression per the revised IWG Response Criteria for Malignant Lymphoma or death from any cause.

“Disease progression” or “progressive disease,” which can be abbreviated as PD, as used herein, refers to a worsening of one or more symptom associated with a particular disease. For example, disease progression for a subject afflicted with a cancer can include an increase in the number or size of one or more malignant lesions, tumor metastasis, and death.

The “duration of response,” which can be abbreviated as DOR, as used herein refers to the period of time between a subject's first objective response to the date of confirmed disease progression, per the revised IWG Response Criteria for Malignant Lymphoma, or death.

The term “overall survival,” which can be abbreviated as OS, is defined as the time from the date of treatment to the date of death.

The term “immune cell” as used herein refers to any cell of the human immune system. The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). T-cell receptors (TCR) differentiate T cells from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory Tscm cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory T_{EM} cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. B-cells, on the other hand, play a principal role in humoral immunity (with antibody involvement). A B cell makes antibodies and antigens and performs the role of antigen-presenting cells (APCs) and turns into memory B-cells after activation by antigen interaction. In mammals, immature B-cells are formed in the bone marrow, where its name is derived from.

The terms “modified” and “mutated,” when used herein to refer to a nucleotide or amino acid sequence, refers to a change in the sequence relative to a wild-type sequence or a specified reference sequence. The terms “modified” and “mutated” do not require a step in a process for making the modified or mutated sequence (e.g., the modified beta chain sequence), unless otherwise specified. Rather, these terms indicate that there is a variation in the modified or mutated sequence relative to a reference sequence, e.g., a wild-type sequence.

The term “any amino acid,” as used herein, means any known amino acid. Amino acids are organic compounds comprising (i) an amine (—NH₂) functional group, (ii) a carboxyl (—COOH) functional group, and (iii) a side chain (R group), wherein the side chain is specific to each amino acid. This includes but is not limited to any naturally occurring amino acid, as well as any modifications and variants thereof. There are about 500 naturally occurring amino acids, 20 of which are encoded by the genetic code. Amino acids with positively charged side chains include arginine (Arg; R), histidine (His, H), and lysine (Lys; K). Amino acids with a negatively charged side chain include aspartic acid (Asp; D) and glutamic acid (Glu; E). Amino acids with a polar uncharged side chain include serine (Ser; S), threonine (Thr; T), glutamine (Gln; Q), and asparagine (Asn; N). Amino acids with a hydrophobic side chain include alanine (Ala; A), isoleucine (Ile; I), leucine (Leu; L), methionine (Met; M), phenylalanine (Phe; F), valine (Val; V), Tryptophan (Trp; W), Tyrosine (Tyr; Y). Tryptophan (Trp; W), tyrosine (Tyr; Y), and methionine (Met; M) can also be classified as polar and/or amphipathic, in that these amino acids can often be found at the surface of proteins or lipid membranes. Additional amino acids include cysteine (Cys; C), selenocysteine (Sec; U), glycine (Gly; G) and proline (Pro; P).

The term “genetically engineered” or “engineered” refers to a method of modifying the genome of a cell, including, but not limited to, deleting a coding or non-coding region or a portion thereof or inserting a coding region or a portion thereof. In some aspects, the cell that is modified is a lymphocyte, e.g., a T cell or a modified cell that expresses CD4, which can either be obtained from a patient or a donor. The cell can be modified to express an exogenous construct, such as, e.g., a T cell receptor (TCR) disclosed herein, which is incorporated into the cell's genome. In some aspects, the cell is modified to express CD4. Any method of genetic engineering can be used in the compositions and methods disclosed herein. In some aspects, the genome of a modified immune cell disclosed herein is genetically engineered using CRISPR technology.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT), and allogeneic T cell transplantation.

Cells used in an immunotherapy described herein can come from any source. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “conditioning” and “pre-conditioning” are used interchangeably herein and indicate preparing a patient in need of an immune cells, e.g., a T cell, therapy for a suitable condition. Conditioning as used herein includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy. In one aspect, “conditioning” comprises increasing a serum level of one or more cytokines, e.g., interleukin 7 (IL-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof. In another aspect, “conditioning” comprises increasing a serum level of IL-7, IL-15, IP-10, MCP-1, PLGF, CRP, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Various aspects of the invention are described in further detail in the following subsections.

### II. Methods of the Disclosure

The present disclosure is directed to methods of treating a subject in need thereof, comprising administering to the subject a population of modified immune cells, which comprises one or more modified immune cells having decreased expression of one or more components of the SAGA (Spt-Ada-Gcn5-acetyltransferase) complex, relative to an unmodified immune cell. In some aspects, the population of modified immune cells are modified by culturing the immune cells ex vivo under conditions that block or reduce the expression of one or more components of the SAGA complex prior to administration to the subject. Some aspects of the present disclosure are directed to methods of treating a subject in need thereof, comprising (i) culturing a population of immune cells ex vivo under conditions that block or reduce the expression of one or more components of the SAGA thereby generating a population of modified immune cells, and (ii) administering the population of modified immune cells to the subject.

In some aspects, the population of immune cells comprises CD8+ T cells. In some aspects, the population of immune cells comprises one or immune cells selected from the group consisting of T cells, natural killer (NK) cells, B cells, regulatory T (Treg) cells, tumor infiltrating lymphocytes, and any combination thereof. In some aspects, the population of immune cells comprises a stem cell-like memory T (T_{SCM}) cell. In some aspects, the population of immune cells comprises a double negative T (DNT) cell.

In some aspects, the modified immune cells disclosed herein exhibit increased persistence relative to unmodified immune cells, e.g., immune cells having normal expression of SAGA complex components. In some aspects, the modified immune cells exhibit increased expression of one or more activation marker following re-stimulation, relative to unmodified immune cells following re-stimulation. In some aspects, the activation marker is selected from the group consisting of CD25, HLA-DR, CD69, and any combination thereof. In some aspects, the modified immune cells exhibit increased expression of CD25, following re-stimulation, relative to unmodified immune cells following re-stimulation. In some aspects, the modified immune cells exhibit increased expression of HLA-DR, following re-stimulation, relative to unmodified immune cells following re-stimulation. In some aspects, the modified immune cells exhibit increased expression of CD69, following re-stimulation, relative to unmodified immune cells following re-stimulation. In some aspects, the re-stimulation comprises contacting the cells with CD3, CD28,CD2, or any combination thereof. In some aspects, the re-stimulation is applied at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after an initial stimulation.

In some aspects, the modified immune cells exhibit increased expression of one or more activation marker following serial stimulation, relative to unmodified immune cells following serial stimulation. In some aspects, the activation marker is selected from the group consisting of CD25, HLA-DR, CD69, and any combination thereof. In some aspects, the modified immune cells exhibit increased expression of CD25, following serial stimulation, relative to unmodified immune cells following serial stimulation. In some aspects, the modified immune cells exhibit increased expression of HLA-DR, following serial stimulation, relative to unmodified immune cells following serial stimulation. In some aspects, the modified immune cells exhibit increased expression of CD69, following serial stimulation, relative to unmodified immune cells following serial stimulation. In some aspects, the serial stimulation comprises a first stimulation, a first re-stimulation, and a second re-stimulation. In some aspects, the serial stimulation further comprises a third re-stimulation. In some aspects, the serial stimulation further comprises a fourth re-stimulation. In some aspects, the modified immune cells exhibit increased expression of one or more activation markers after serial stimulation, wherein the one or more activation markers are selected from the group consisting of CD25, HLA-DR, CD69, and any combination thereof at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, or at least about 41 days after an initial stimulation, relative to unmodified immune cells following serial stimulation.

In some aspects, the expression of the one or more activation markers is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to unmodified immune cells following re-stimulation and/or serial stimulation.

In some aspects, the modified immune cells exhibit increased expression of CD107a following re-stimulation, relative to unmodified cells following re-stimulation. In some aspects, the modified immune cells exhibit increased expression of CD107a following serial stimulation, relative to unmodified cells following serial stimulation. In some aspects, the expression of CD107a is increased by at least about 10%, at least about 15%, at least about 20%, at least about 25%, at least about 30%, at least about 40%, at least about 50%, at least about 75%, or by at least about 100%, relative to unmodified cells following re-stimulation and/or serial stimulation.

In some aspects, the modified immune cells exhibit increased phospho-MEK levels following stimulation, relative to unmodified cells following stimulation. In some aspects, the level of phospho-MEK is increased by at least about 10%, at least about 20%, at least about 25%, at least about 30%, at least about 40%, at least about 50%, at least about 75%, at least about 100%, at least about 125%, at least about 150%, at least about 175%, at least about 200%, at least about 250%, at least about 300%, at least about 350%, at least about 400%, at least about 450%, at least about 500% relative to unmodified cells following re-stimulation and/or serial stimulation.

In some aspects, the modified immune cells exhibit increased phospho-AKT levels following stimulation, relative to unmodified cells following stimulation. In some aspects, the level of phospho-AKT is increased by at least about 10%, at least about 20%, at least about 25%, at least about 30%, at least about 40%, at least about 50%, at least about 75%, at least about 100%, at least about 125%, at least about 150%, at least about 175%, at least about 200%, at least about 250%, at least about 300%, at least about 350%, at least about 400%, at least about 450%, at least about 500% relative to unmodified cells following re-stimulation and/or serial stimulation.

In some aspects, the modified immune cells exhibit decreased expression of one or more T cell exhaustion markers following re-stimulation, relative to unmodified immune cells following re-stimulation. In some aspects, the modified immune cells exhibit decreased expression of one or more T cell exhaustion markers following serial stimulation, relative to unmodified immune cells following serial stimulation. In some aspects, the modified immune cells exhibit decreased expression of LAG3 following re-stimulation, relative to unmodified immune cells following re-stimulation. In some aspects, the modified immune cells exhibit decreased expression of LAG3 following serial stimulation, relative to unmodified immune cells following serial stimulation. In some aspects, the modified immune cells exhibit decreased expression of TIM3 following re-stimulation, relative to unmodified immune cells following re-stimulation. In some aspects, the modified immune cells exhibit decreased expression of TIM3 following serial stimulation, relative to unmodified immune cells following serial stimulation.

In some aspects, the expression of the one or more T cell exhaustion markers (e.g., LAG3 and/or TIM3) is decreased by at least about 10%, at least about 15%, at least about 20%, at least about 25%, at least about 30%, at least about 40%, at least about 50%, or at least about 75%, relative to unmodified cells following re-stimulation and/or serial stimulation.

In some aspects, the subject is a human. In some aspects, the subject is afflicted with a cancer. In some aspects, the cancer is selected from the group consisting of melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of the cancers. In some aspects, the cancer is relapsed. In some aspects, the cancer is refractory. In some aspects, the cancer is advanced, e.g., locally advanced. In some aspects, the cancer is metastatic.

In some aspects, the subject is afflicted with an infectious disease. In some aspects, the infectious disease is selected from HIV, hepatitis B infections (HBV), cytomegalovirus (CMV), Epstein-Barr virus (EBV), an Aspergillus fungal infection, and any combination thereof. In some aspects, the subject is afflicted with autoimmune disease. In some aspects the autoimmune disease is selected from systemic lupus erythematosus (SLE), pemphigus vulgaris (PV), Multiple sclerosis (MS), colitis, and any combination thereof. In some aspects, the subject is afflicted with cardiac fibrosis. In some aspects, the subject is afflicted with liver fibrosis. In some aspects, the subject is afflicted with mesothelioma. In some aspects, the subject is afflicted with cellular senescence.

### II.A. The SAGA Complex

The SAGA (Spt-Ada-Gcn5-acetyltransferase) complex is an evolutionary conserved, multifunctional co-activator comprising nineteen subunits. It is organized into separate modules with distinct activities, containing a structural core, a histone acetyltransferase (HAT), a histone deubiquitinase (DUB), and an activator-binding module. SAGA and its related complexes are involved in several distinct signaling pathways, mostly through stimulating transcription via two chromatin-modifying enzymatic modules and by delivering the TATA box binding protein (TBP) to nucleate the pre-initiation complex on DNA.

Transcription of protein-encoding genes begins with the formation of a pre-initiation complex (PIC) comprising RNA polymerase II and several general transcription factors. In addition to SAGA, another multiprotein complex, transcription factor IID (TFIID), can deliver TBP to gene promoters, and is required for global gene expression in yeast. Recent studies have indicated a more specific role for SAGA than for TFIID. For example, SAGA-dominated promoters tend to have a consensus TATA box, are more stress-regulated/inducible genes, and tend to be more tightly regulated. Upon deletion of SPT3, a TBP-interacting subunit of SAGA, levels of total mRNA were reduced for only about 10% of yeast genes compared to those regulated by TBP-associated factor 1 (Taf1), a subunit of the TFIID which reduced for roughly 90% of yeast genes. This led to the distinction between two different gene classes: (1) the SAGA-dominated genes, which are positively regulated by Spt3, but are essentially independent of Taf1, and (2) the larger class (90%) of TFIID-dominated genes, which are more dependent on Taf1 than on Spt3. Therefore, as a general model, it was proposed that TBP recruitment is primarily dependent on SAGA at TATA-containing promoters but dominated by TFIID at the TATA-like (or TATA-less) promoters. Nonetheless, the central module has been shown to be structurally related to TFIID, suggesting that TBP binding in both complexes shares some common features.

Some data suggest a compensatory increase of the half-life of a majority of mRNAs upon SAGA depletion, explaining the limited changes in steady-state mRNA levels in the different SAGA mutant strains. Therefore, the decrease in Pol II transcription following SAGA depletion was compensated by increasing mRNA half-lives, as previously reported for mutations in Pol II and Mediator, or inhibition of the kinase activity in TFIIH. These studies show the importance of SAGA in initiating transcription, and point to mechanisms of compensation when components of the SAGA complex are lost.

The expression of any one or more components of the SAGA complex can be blocked or reduced in the compositions and methods disclosed herein. In some aspects, the expression of one or more of USP22, TADA1, TADA2b, and TAF6L is blocked or reduced. In some aspects, a population of modified immune cells of the present disclosure has decreased ablated expression of USP22. In some aspects, a population of modified immune cells of the present disclosure has decreased ablated expression of TADA1. In some aspects, a population of modified immune cells of the present disclosure has decreased ablated expression of TADA2b. In some aspects, a population of modified immune cells of the present disclosure has decreased ablated expression of TAF6L.

In some aspects, a population of modified immune cells of the present disclosure has decreased ablated expression of USP22 and an additional component of the SAGA complex. In some aspects, a population of modified immune cells of the present disclosure has decreased ablated expression of USP22 and TADA1. In some aspects, a population of modified immune cells of the present disclosure has decreased ablated expression of USP22 and TADA2b. In some aspects, a population of modified immune cells of the present disclosure has decreased ablated expression of USP22 and TAF6L.

In some aspects, a population of modified immune cells of the present disclosure has decreased ablated expression of TADA1 and an additional component of the SAGA complex. In some aspects, a population of modified immune cells of the present disclosure has decreased ablated expression of TADA1 and TADA2b. In some aspects, a population of modified immune cells of the present disclosure has decreased ablated expression of TADA1 and TAF6L.

In some aspects, a population of modified immune cells of the present disclosure has decreased ablated expression of TADA2b and an additional component of the SAGA complex. In some aspects, a population of modified immune cells of the present disclosure has decreased ablated expression of TADA2b and TAF6L. In some aspects, a population of modified immune cells of the present disclosure has decreased ablated expression of TAF6L and an additional component of the SAGA complex.

In some aspects, a population of modified immune cells of the present disclosure has decreased ablated expression of USP22, TADA1, and TADA2b. In some aspects, a population of modified immune cells of the present disclosure has decreased ablated expression of USP22, TADA1, and TAF6L. In some aspects, a population of modified immune cells of the present disclosure has decreased ablated expression of TADA1, TADA2b, and TAF6L. In some aspects, a population of modified immune cells of the present disclosure has decreased ablated expression of USP22, TADAI, TADA2b, and TAF6L.

Expression of the one or more components of the SAGA complex can be inhibited, decreased, reduced, and/or blocked by any method. In some aspects, the expression of the one or more components of the SAGA complex is inhibited, decreased, reduced, and/or blocked by contacting the immune cell with an antisense oligonucleotide, wherein the antisense oligonucleotide hybridizes with a transcript encoding the component of the SAGA complex. In some aspects, the expression of the one or more components of the SAGA complex is inhibited, decreased, reduced, and/or blocked by contacting the immune cell with a siRNA, wherein the siRNA hybridizes with a transcript encoding the component of the SAGA complex. In some aspects, the expression of the one or more components of the SAGA complex is inhibited, decreased, reduced, and/or blocked by contacting the immune cell with a shRNA, wherein the shRNA hybridizes with a transcript encoding the component of the SAGA complex.

In some aspects, the expression of the one or more components of the SAGA complex is inhibited, decreased, reduced, and/or blocked by contacting the immune cell with small molecule inhibitor. Any small molecule inhibitor can be used in the methods disclosed herein. Small molecule inhibitors for use in the methods provided herein include, but are not limited to GSK4027, L-Moses inhibitor (inhibitors of KAT2A), broad spectrum histone deacetylase inhibitors (e.g., trichostatin A), antineoplastic agents (e.g., pirarubicin), and combinations thereof.

In some aspects, the expression of the one or more components of the SAGA complex is inhibited, decreased, reduced, and/or blocked by prime editing, wherein the immune cell is contacted with a prime editing guide RNA (pegRNA), wherein the pegRNA hybridizes with a transcript encoding the component of the SAGA complex.

In some aspects, the expression of the one or more components of the SAGA complex is inhibited, decreased, reduced, and/or blocked by contacting the immune cell with contacting the immune cells with a Cas9 protein and a CRISPR-cas system guide RNA (gRNA). In some aspect, T cells isolated from healthy donors are subjected to electroporation with individual Cas9 ribonucleoproteins (RNPs) to achieve single-target-gene knockout in the T cells. Efficiency and level of knockout may be assessed through western blotting and flow cytometry (where possible) in order to measure protein level of these targets compared to wild type control. SAGA activity can be evaluated by measuring global H2B ubiquitination and H3 acetylation levels by western blot. In some aspects, the activity of the SAGA gene regulation complex is decreased by at 30 least 70%, at least 80%, at least 90% or at least 99% in a T cell population treated according to methods described herein.

In some aspects, the gRNA is a single guide RNA (sgRNA). In some aspects, the Cas9 protein, the gRNA, or both are expressed from a heterologous expression construct introduced into the immune cell. In some aspects, the gRNA, the antisense oligonucleotide, the shRNA, the siRNA, the pegRNA, or any combination thereof are expressed from a heterologous expression construct introduced into the immune cell. In some aspects, the gRNA, the antisense oligonucleotide, the shRNA, the siRNA, the pegRNA, or any combination thereof are contacted with the immune cell exogenously. In some aspects, the contacting of the immune cells with the gRNA, the antisense oligonucleotide, the shRNA, the siRNA, the pegRNA, or any combination thereof knocks out the expression of the target component of the SAGA complex in the immune cell.

In some aspects, the gRNA, the antisense oligonucleotide, the shRNA, the siRNA, the pegRNA, or any combination thereof specifically hybridizes to a target DNA region within a coding region encoding a component of the SAGA complex selected from the group consisting of USP22, TADA1, TADA2b, and TAF6L. In some aspects, the gRNA, the antisense oligonucleotide, the shRNA, the siRNA, the pegRNA, or any combination thereof specifically hybridizes to a target DNA region within the USP22 coding region. In some aspects, the gRNA, the antisense oligonucleotide, the shRNA, the siRNA, the pegRNA, or any combination thereof specifically hybridizes to a target DNA region within the TADAI coding region. In some aspects, the gRNA, the antisense oligonucleotide, the shRNA, the siRNA, the pegRNA, or any combination thereof specifically hybridizes to a target DNA region within the TADA2b coding region. In some aspects, the gRNA, the antisense oligonucleotide, the shRNA, the siRNA, the pegRNA, or any combination thereof specifically hybridizes to a target DNA region within the TAF6L coding region.

In some aspects, the method comprises contacting the immune cell with (i) a first gRNA (or antisense oligonucleotide, shRNA, siRNA, or pegRNA) and (ii) a second gRNA (or antisense oligonucleotide, shRNA, siRNA, or pegRNA); wherein the first gRNA (or antisense oligonucleotide, shRNA, siRNA, or pegRNA) hybridizes to a target DNA region within a coding region encoding a first component of the SAGA complex (or a transcript thereof), wherein the second gRNA (or antisense oligonucleotide, shRNA, siRNA, or pegRNA) hybridizes to a target DNA region within a coding region encoding a second component of the SAGA complex (or a transcript thereof), and wherein the first component of the SAGA complex and the second component of the SAGA complex are different. In some aspects, the first component of the SAGA complex is USP22, and the second component of the SAGA complex is TADA1. In some aspects, the first component of the SAGA complex is USP22, and the second component of the SAGA complex is TADA2b. In some aspects, the first component of the SAGA complex is USP22, and the second component of the SAGA complex is TAF6L. In some aspects, the first component of the SAGA complex is TADA1, and the second component of the SAGA complex is TADA2b. In some aspects, the first component of the SAGA complex is TADA1, and the second component of the SAGA complex is TAF6L. In some aspects, the first component of the SAGA complex is TADA2b, and the second component of the SAGA complex is TAF6L.

In some aspects, the method further comprises contacting the immune cell with a third gRNA (or antisense oligonucleotide, shRNA, siRNA, or pegRNA), wherein the third gRNA (or antisense oligonucleotide, shRNA, siRNA, or pegRNA) hybridizes to a target DNA region within a coding region encoding a third component of the SAGA complex (or a transcript thereof); and wherein the first component of the SAGA complex, the second component of the SAGA complex, and the third component of the SAGA complex are different. In some aspects, the first component of the SAGA complex is USP22, the second component of the SAGA complex is TADA1, and the third component of the SAGA complex is TADA2b. In some aspects, the first component of the SAGA complex is USP22, the second component of the SAGA complex is TADAI, and the third component of the SAGA complex is TAF6L. In some aspects, the first component of the SAGA complex is TADAI, the second component of the SAGA complex is TADA2b, and the third component of the SAGA complex is TAF6L. In some aspects, the first component of the SAGA complex is USP22, the second component of the SAGA complex is TADA2b, and the third component of the SAGA complex is TAF6L.

In some aspects, the method further comprises contacting the immune cell with a fourth gRNA (or antisense oligonucleotide, shRNA, siRNA, or pegRNA), wherein the fourth gRNA (or antisense oligonucleotide, shRNA, siRNA, or pegRNA) hybridizes to a target DNA region within a coding region encoding a fourth component of the SAGA complex (or a transcript thereof); and wherein the first component of the SAGA complex, the second component of the SAGA complex, the third component of the SAGA complex are different, and the fourth component of the SAGA complex are different. In some aspects, the first component of the SAGA complex is USP22, the second component of the SAGA complex is TADA1, the third component of the SAGA complex is TADA2b, and the fourth component of the SAGA complex is TAF6L.

### II.B. Immune Cells

Some aspects of the present disclosure are directed to methods of treating a subject in need thereof, comprising administering to the subject a population of modified immune cells, which comprises one or more modified immune cells having decreased expression of one or more components of the SAGA (Spt-Ada-Gcn5-acetyltransferase) complex, relative to an unmodified immune cell. Any immune cell can be used in the methods disclosed herein. In some aspects, the immune cell is obtained from the subject to which the modified immune cells will be delivered. In some aspects, the immune cells are obtained from a different subject. In some aspects, the immune cells are derived from a pluripotent stem cell, e.g., an embryonic stem cell (ESC), a hematopoietic stem cell (HSC), or an induced pluripotent stem cell (iPSC). In some aspects, the immune cell is isolated from peripheral blood mononuclear cells (PBMCs). In some aspects, the immune cell is isolated from a tumor biopsy, e.g., a tumor infiltrating lymphocyte (TIL).

In some aspects, the population of immune cells comprises an immune cell selected from the group consisting of a T cell, a TsCM cell, a double negative T (DNT) cell, a natural killer (NK) cell, a B cells, a regulatory T (Treg) cell, a tumor infiltrating lymphocyte, and any combination thereof. In some aspects, the population of immune cells comprises CD8+ T cells.

In some aspects, the population of immune cells comprises one or more immune cells that further comprise a T cell receptor (TCR). In some aspects, the TCR comprises an antigen-binding domain that binds a tumor antigen. In some aspects, the tumor antigen is selected from the group consisting of NYESO-1, MAGE-A1, MAGE-A2, MAGE-A3, MAGE-A4, MAGE-A6, MAGE-A8, MAGE-A9, MAGE-A10, MAGE-A12, MART-1, gp100, WT1, tyrosinase, PRAME, p53, HPV-E6, HBV, TRAIL&DR4, thyroglobulin, TGFβII frameshift antigen, LAGE-1A, KRAS G12V, HPV-E7, HERV-E, HA-1, CMV, CEA, AFP, and any combination thereof. In some aspects, the TCR comprises an antigen-binding domain that binds NYESO-1. In some aspects, the TCR is an αβ TCR. In some aspects, the TCR is a γδ TCR.

In some aspects, the population of immune cells comprises one or more immune cells that further comprise a chimeric antigen receptor (CAR). In some aspects, the CAR comprises an antigen-binding domain that specifically binds a tumor antigen. In some aspects, the tumor antigen is selected from the group consisting of CD19, BCMA, CD30, CD33, CD123, FLT3, and any combination thereof.

### II.C. Combination Therapies

Some aspects of the present disclosure are directed to methods of treating a subject in need thereof, comprising administering to the subject (i) a population of modified immune cells, which comprises one or more modified immune cells having decreased expression of one or more components of the SAGA (Spt-Ada-Gcn5-acetyltransferase) complex, relative to an unmodified immune cell; and (ii) an additional anticancer agent. Any anticancer agent can be used in the methods disclosed herein. In some aspects, the anticancer agent acts by killing cancer cells. In some aspects, the anticancer agent acts by stimulating an immune response. In some aspects, the population of modified immune cells disclosed herein are administered concurrently with the additional anticancer agent. In some aspects, the population of modified immune cells disclosed herein and the additional anticancer agent are administered sequentially (e.g., on the same day or on different days).

In some aspects, the additional anticancer agent comprises an immunotherapy, a chemotherapy, a cytokine, a radiation therapy, a surgery, or any combination thereof.

In some aspects, the additional anticancer agent comprises an immunotherapy. Any immunotherapy can be used in combination with the methods disclosed herein. In some aspects, the additional anticancer agent comprises a cell based immunotherapy. In some aspects, the additional anticancer agent comprises an antibody or an antigen-binding portion thereof.

In some aspects, the additional anticancer agent comprises an antagonist (inhibitor or blocking agent) of a protein that inhibits T cell activation (e.g., immune checkpoint inhibitors), such as CTLA-4, PD-1, PD-L1, PD-L2, GITR, LAG-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, mesothelin, CD27, CD96, TIM-1, TIM-3, and TIM-4. In some aspects, the additional anticancer agent comprises a PD-1 inhibitor, a PD-L1 inhibitor, a CTLA-4 inhibitor, a LAG3 inhibitor, a TIM3 inhibitor, or any combination thereof.

In some aspects, the additional anticancer agent comprises an antibody or an antigen-binding portion thereof that specifically binds PD-1 or PD-L1. In some aspects, the additional anticancer agent comprises an anti-PD-1 antibody selected from nivolumab (OPDIVO®) and pembrolizumab (KEYTRUDA®). In some aspects, the additional anticancer agent is selected from YERVOY® (ipilimumab) or Tremelimumab (to CTLA-4), galiximab (to B7.1), BMS-936558 (to PD-1), MK-3475 (to PD-1), atezolizumab (TECENTRIQ®), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), hμMAbOX40L (to OX40L), Atacicept (to TACI), CP-870893 (to CD40), Lucatumumab (to CD40), Dacetuzumab (to CD40), Muromonab-CD3 (to CD3); anti-GITR antibodies MK4166, TRX518, Medi1873, INBRX-110, LK2-145, GWN-323, GITRL-Fc, and any combination thereof.

In some aspects, the additional anticancer agent comprises an agent that targets (or binds specifically to) a member of the B7 family of membrane-bound ligands that includes B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor or ligand binding specifically to a B7 family member. In some aspects, the additional anticancer agent comprises an agonist of a protein that stimulates T cell activation, such as B7-1, B7-2, CD28, 4-1BB (CD137), 4-1BBL, GITR, ICOS, ICOS-L, OX40, OX40L, CD70, CD27, CD40, DR3 and CD28H. In some aspects, the additional anticancer agent comprises an antagonist of an inhibitory receptor on NK cells or an agonist of an activating receptor on NK cells, e.g., an antagonist of KIR (e.g., lirilumab).

In some aspects, the additional anticancer agent comprises a treatment selected from irradiation and/or chemotherapy, e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, or camptothecin+apo21/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002;8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al, Cancer Research 2005;65:4799-808), c-FLIP (cellular FLICE-inhibitory protein) modulators (e.g., natural and synthetic ligands of PPARy (peroxisome proliferator-activated receptor γ), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3P inhibitors, IAP inhibitors and/or genotoxic drugs.

In some aspects, the additional anticancer agent comprises one or more anti-proliferative cytotoxic agents. In some aspects, the additional anticancer agent comprises an alkylating agents (including, without limitation, nitrogen mustards, ethylenimine derivatives, alkyl sulfonates, nitrosoureas and triazenes). In some aspects, the additional anticancer agent comprises uracil mustard, chlormethine, cyclophosphamide (CYTOXANR) fosfamide, melphalan, chlorambucil, pipobroman, triethylenemelamine, triethylenethiophosphoramine, busulfan, carmustine, lomustine, streptozocin, dacarbazine, temozolomide, and any combination thereof.

In some aspects, the additional anticancer agent comprises an antimetabolite (including, without limitation, folic acid antagonists, pyrimidine analogs, purine analogs and adenosine deaminase inhibitors). In some aspects, the additional anticancer agent comprises methotrexate, 5-fluorouracil, floxuridine, cytarabine, 6-mercaptopurine, 6-thioguanine, fludarabine phosphate, pentostatine, gemcitabine, and any combination thereof.

In some aspects, the additional anticancer agent comprises a taxane, paclitaxel (e.g., TAXOL™M), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone Bl, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone BIO, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5 (10)-trien-3-ol, cyclostreptin, isolaulimalide, laulimalide, 4-epi-7-dehydroxy-14,16-didemethyl-(+)-discodermolides, and cryptothilone 1, a microtubuline stabilizing, and any combination thereof.

In some aspects, the additional anticancer agent comprises a lymphodepleting chemotherapy. In some aspects, the lymphodepleting chemotherapy is administered prior to the modified immune cells. In some aspects, the lymphodepleting chemotherapy comprises cyclophosphamide. In some aspects, the lymphodepleting chemotherapy comprises fludarabine. In some aspects, the lymphodepleting chemotherapy comprises cyclophosphamide and fludarabine.

Some aspects of the present disclosure are directed to methods of treating a subject in need thereof, comprising administering to the subject (i) a population of modified immune cells, which comprises one or more modified immune cells having decreased expression of one or more components of the SAGA (Spt-Ada-Gcn5-acetyltransferase) complex, relative to an unmodified immune cell; and (ii) a cytokine. In some aspects, the cytokine comprises an interleukin. In some aspects, the cytokine is selected from IL2, IL7, IL12, IL15, IL17, IL21, granulocyte macrophage colony-stimulating factor (GM-CSF), and interferon (IFN)-α. In some aspects, the cytokine comprises IL2.

### EXAMPLES

### Example 1—Generation of CD8+ T Cells with Depleted SAGA Complex

A group of sgRNAs were identified that target various components of the SAGA (Spt-Ada-Gcn5-acetyltransferase) complex among negative regulators of CD8+ T cell proliferation, activation, and effector function. The screen was validated by individually knocking out components of the SAGA complex with Cas9 ribonucleoproteins (RNPs), and an increase in CD107a expression was observed, as well as other markers of cytolytic activity (granzyme B and perforin).

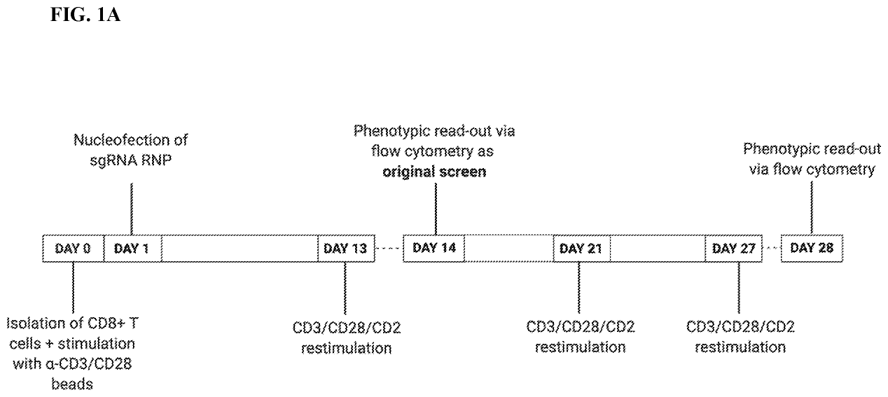

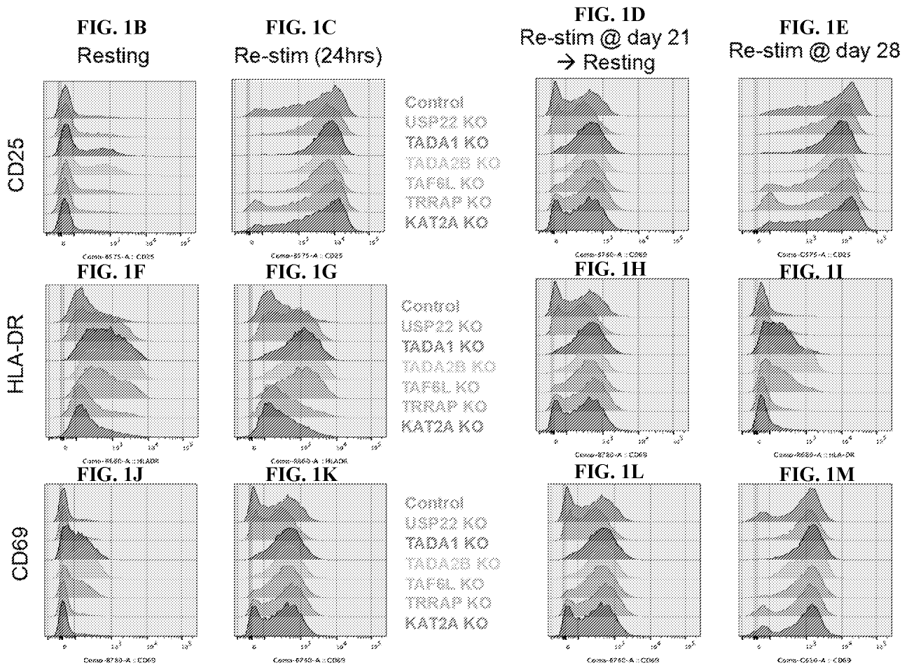

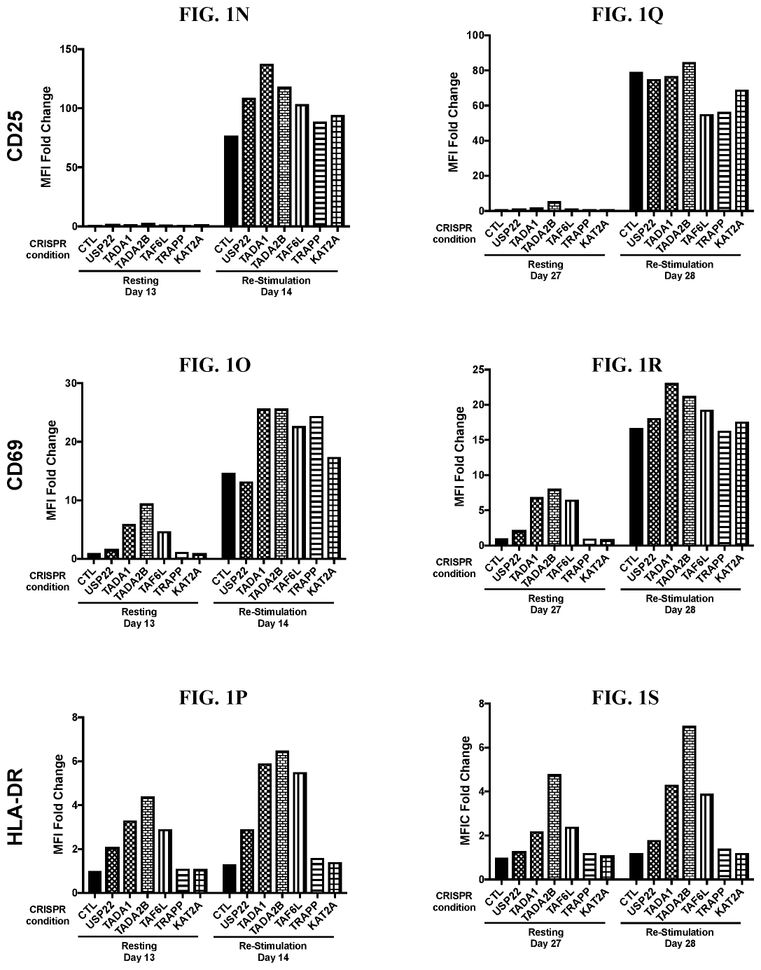

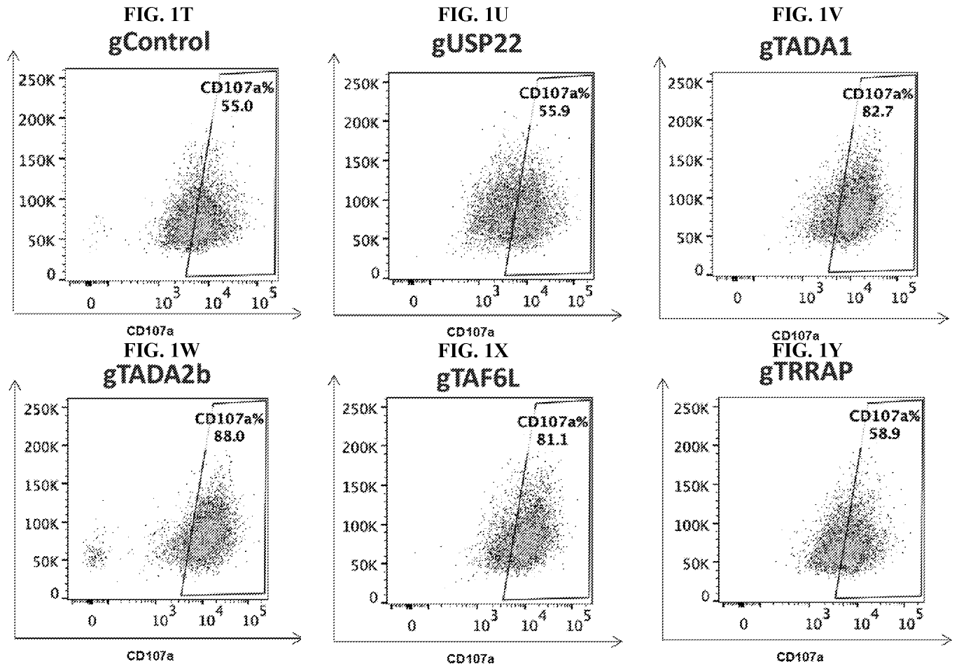

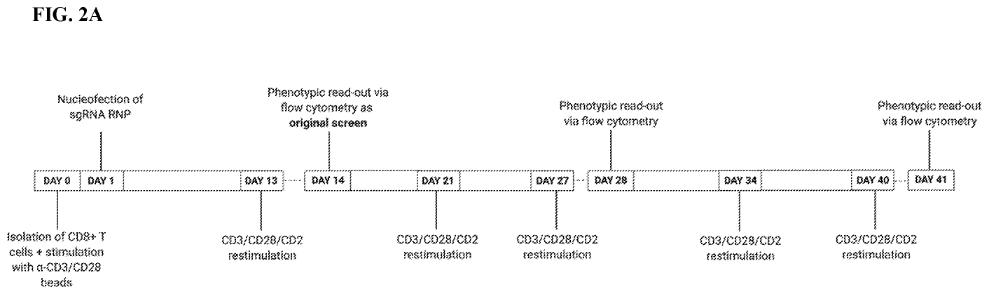

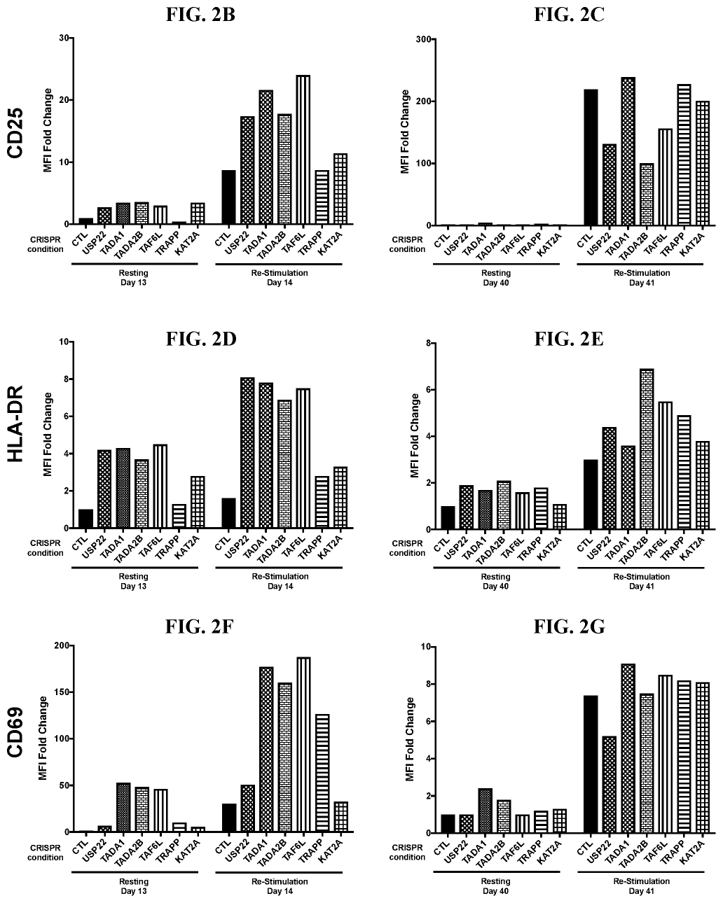

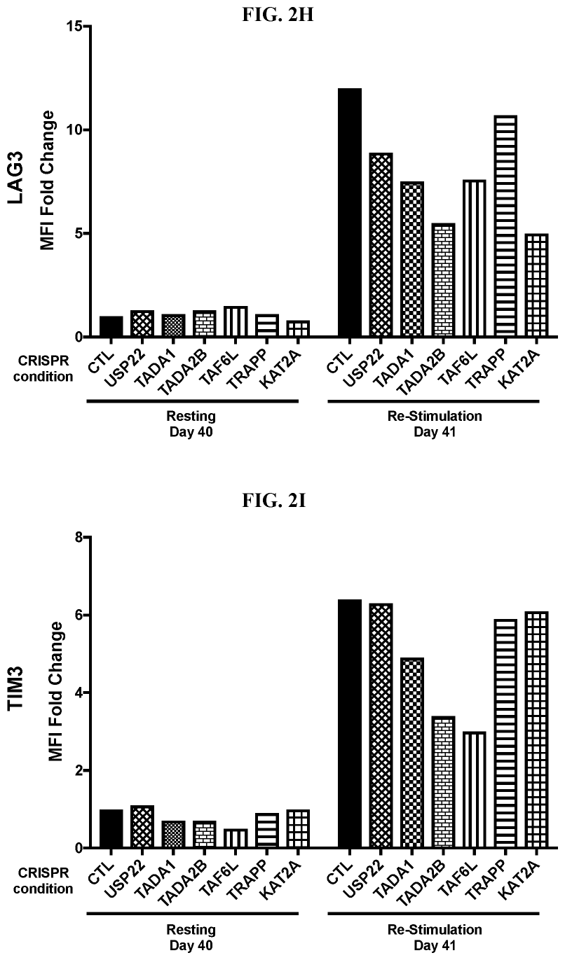

Next, the persistence of CD8+ T cells was assayed following depletion of SAGA complex. Following short (Day 14) and medium term (Day 28) re-stimulation (FIG. 1A), SAGA depleted T cells exhibited higher levels of activation markers CD25 (FIGS. 1B-1E, 1N, and 1Q), HLA-DR (FIGS. 1F-1I, 1P, and 1S), and CD69 (FIGS. 1J-1M, 1O, and 1S). Furthermore, repetitive stimulation for up to 41 days (FIG. 2A) led to a larger portion of SAGA depleted cells to express activation markers compared to control (FIGS. 2B-2G). Remarkably, decreased expression of exhaustion markers, LAG3 and TIM3, was also observed (FIGS. 2H-2I). CD107a expression was also measured at medium term (Day 28) re-stimulation. CD107a expression was increased in SAGA depleted T cells lacking expression of USP22 (FIG. 1U), TADA1 (FIG. 1V), TADA2b (FIG. 1W), TAF6L (FIG. 1X), or TRRAP (FIG. 1Y), as compared to control (wild type; FIG. 1T) cells.

### Example 2—Increased Activity of TCR Transgenic T Cells with Depleted SAGA Complex

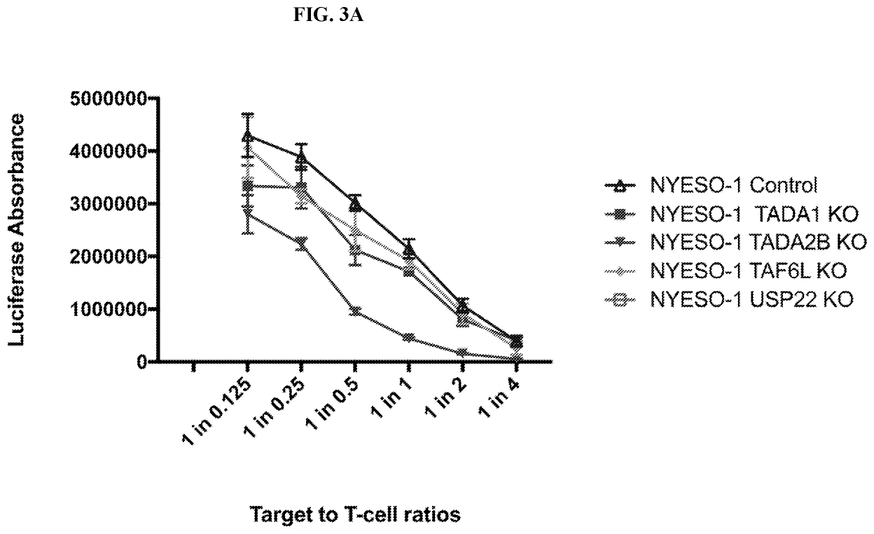

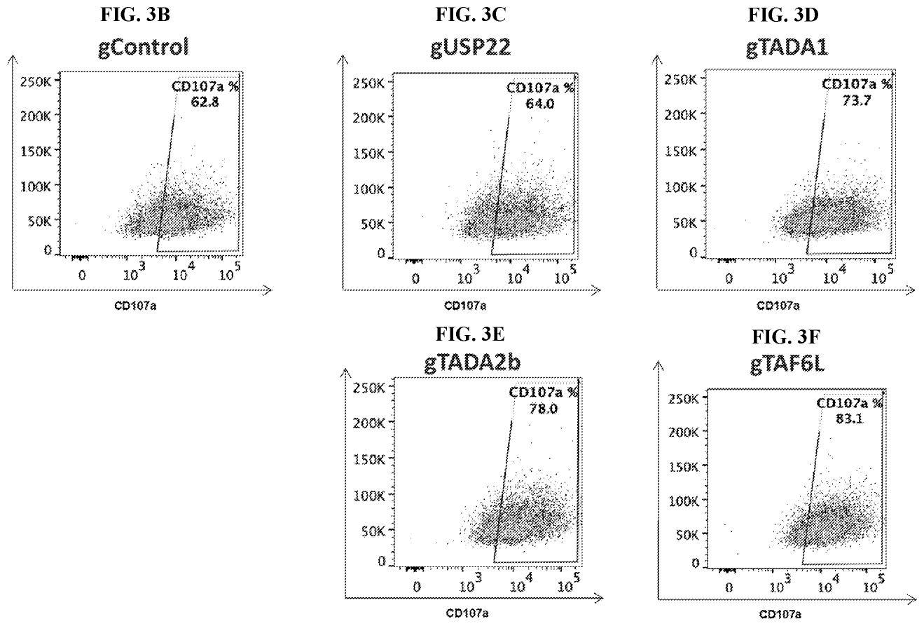

Antigen-specific target cell killing by CD8+ T cells was also improved in T cells having depletion of SAGA complex. A TCR-β and TCR-α pair was generated that recognizes the NY-ESO-1 tumor antigen into the TRAC locus of polyclonal T cells isolated from healthy human donors, thus generating NYESO-1+ transgenic TCR T cells. Co-culture of SAGA depleted antigen-specific T cells with the NY-ESO-1+ melanoma cell line A375 rapidly killed target cancer cells in vitro (FIG. 3A). Expression of CD107a in SAGA depleted NYESO-1+ transgenic TCR T cells was found to increase as compared to control (wild type) cells (FIGS. 3B-3F).

These data provide evidence that depleting the SAGA complex in CD8+ T cells leads to more potent T cell activation, cytolytic activity, antigen-specific killing of cancer cells and longer-term persistence.

### Example 3—CAR-T Cells with Depleted SAGA Complex

CAR-T cells will be generated with and without depletion of SAGA complex. Expression of SAGA complex components will be ablated according to the methods disclosed herein. Briefly, CRISPR guide RNAs targeting USP22, TADAI, TADA2b, TAF6L, or TRRAP will be contacted with T cells to knock out the SAGA complex. T cells will be transduced with an expression vector encoding a CAR construct. Each CAR-T/SAGA depleted cell line will be assayed for persistence, cytolytic activity, and expression of various activation markers, and results will be compared with non-SAGA depleted cells, non-transduced cells, and wild-type cells. Both PBMC isolated T cells and NK cells will be used to generate the CAR-T/SAGA depleted cell lines.

### Example 4—Tumor Infiltrating Lymphocytes with Depleted SAGA Complex

Tumor infiltrating lymphocytes will be isolated from a tumor tissue biopsy. SAGA complex will be depleted according to the methods disclosed herein. Briefly, CRISPR guide RNAs targeting USP22, TADA1, TADA2b, TAF6L, or TRRAP will be contacted with T cells to knock out the SAGA complex. Each SAGA depleted TIL line will be assayed for persistence, cytolytic activity, and expression of various activation markers, and results will be compared with non-SAGA depleted TILs.

### Example 5—In Vivo Analysis of Administered SAGA-Depleted Therapeutic T Cells

A clinical trial will be designed to test the in vivo of T cell therapies comprising administration of SAGA-depleted T cells, as described herein. SAGA-depleted CAR-T, TCR transgenic T cells, and TILs will be administered to human subjects afflicted with a cancer. T cells having no depletion of SAGA complex will be used as a control. Tumor size and growth rate as well as overall survival and progression free survival will be monitored and compared with subjects treated with control T cells.

### Example 6—SAGA-Depleted T Cells Exhibit Increased Activation

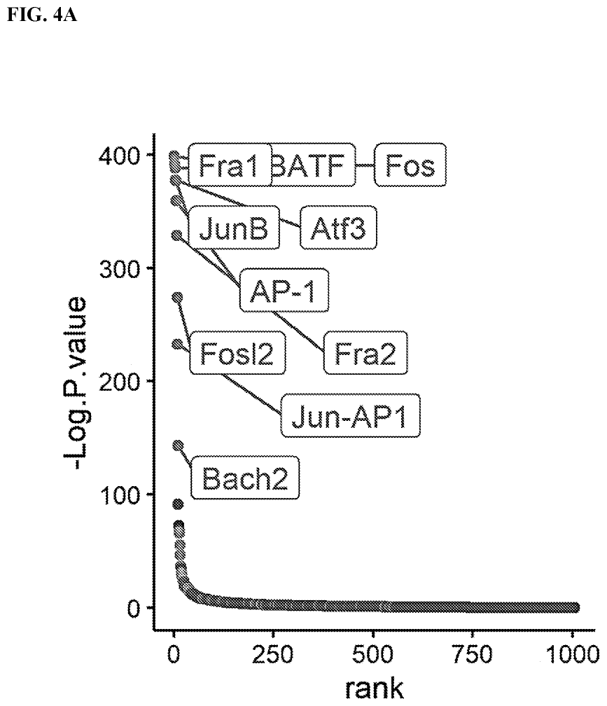

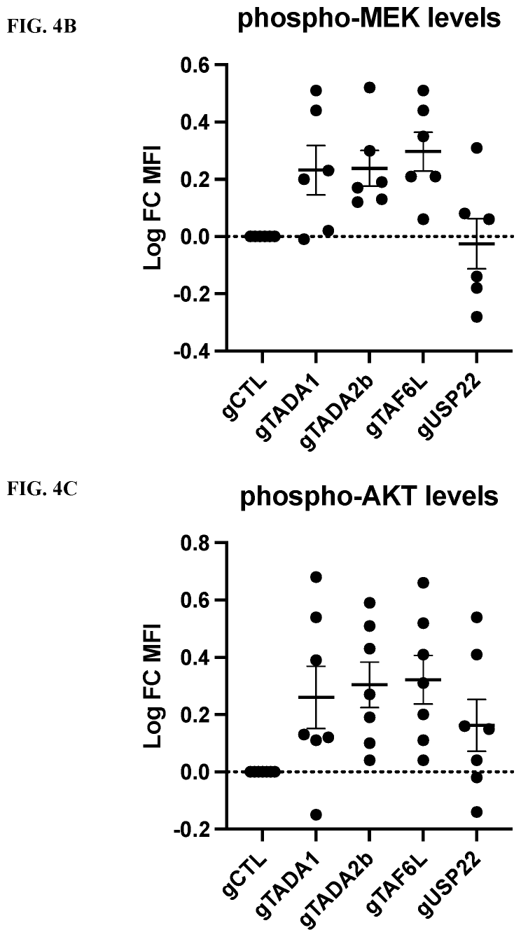

T cells depleted of SAGA were generated by knock out of various targets: gTADA1 KO, gTADA2b KO, gTAF6L KO as well as gControl (˜11 days post KO using CRISPR-cas9). Cell were analyzed by an Assay for Transposase-Accessible Chromatin using sequencing (ATAC-seq). HOMER Analysis of accessibility regions revealed that the top motifs that gained accessibility after SAGA targets KO, but decreased in accessibility in gC_{TL}, belong to canonical transcription factors associated with T cell activation (FIG. 4A).

SAGA-depleted and control T cells were stimulated with phorbol myristate acetate/ionomycin stimulation cocktail for 10 minutes, and MEK and AKT phosphorylation levels were measured. Phospho-MEK (FIG. 4B) and phospho-AKT (FIG. 4C) levels were generally higher in SAGA depleted cells (gTADAI KO, gTADA2b KO, and gTAF6L KO) as compared to control cells, indicating increased activation in SAGA-depleted cells.

### Example 7—SAGA-Depleted T Cells Exhibit Increased Target Tumor Cell Killing In Vitro and In Vivo

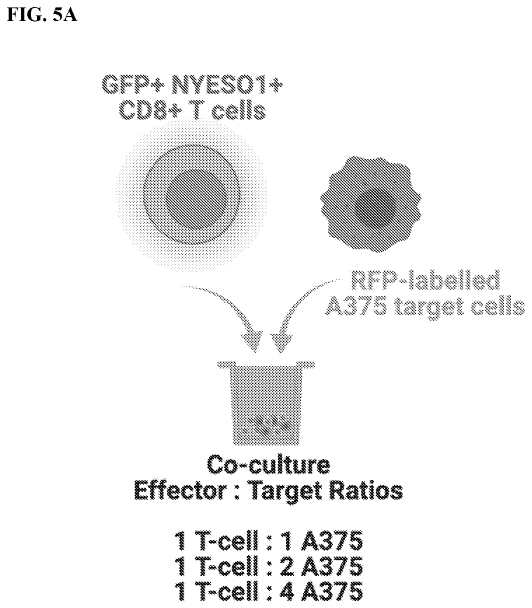

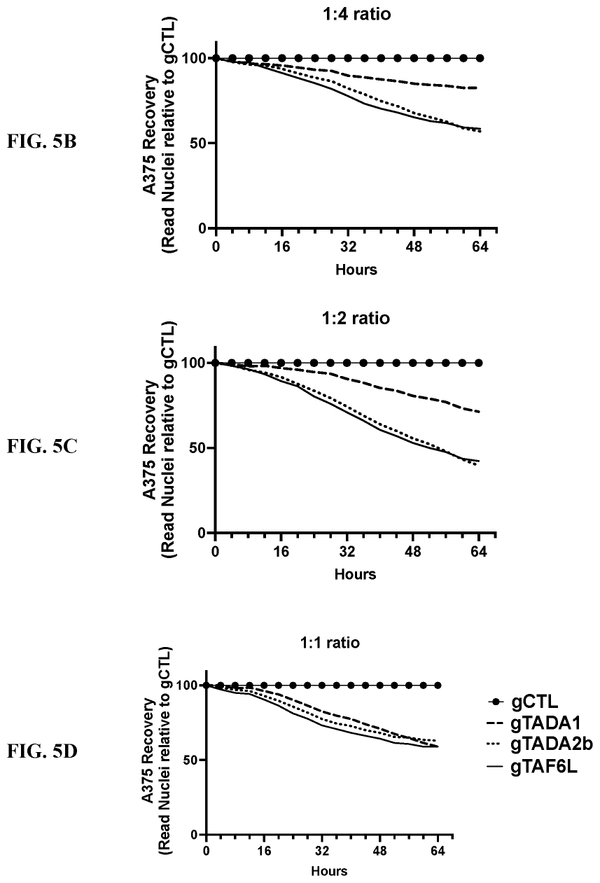

Co-culture assays were conducted to assess the in vitro killing of A375 melanoma cancer cells by transgenic NYESO-1+ T cells depleted of SAGA targets: gTADA1 KO, gTADA2b KO, gTAF6L KO versus gC_{TL} (FIG. 5A). The plates were imaged every 4 h for up to 64 hours using IncuCyte Zoom live-cell imaging (Essen Bioscience).

A375-RFP cell recovery measured by each 4-hour interval shows that transgenic NYESO-1+ T cells depleted of SAGA targets (gTADA1 KO, gTADA2b KO, and gTAF6L KO, as indicated) exhibited increased target tumor cell killing as compared to gC_{TL} at 1:4, 1:2, and 1:1 E:T ratios (FIGS. 5B-5D, respectively).

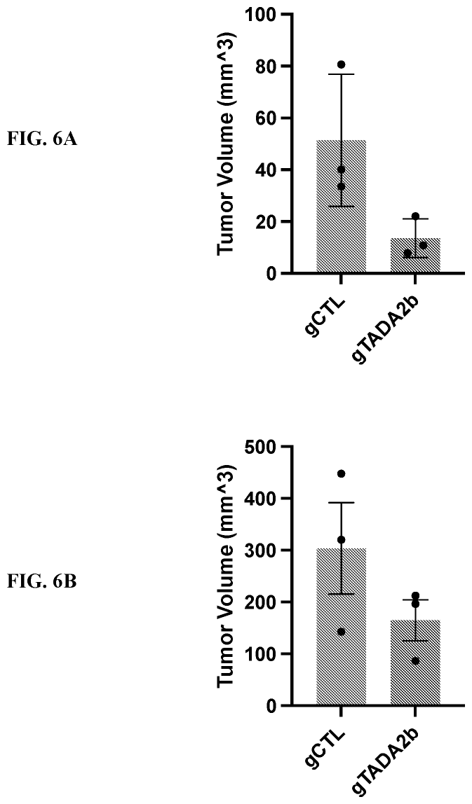

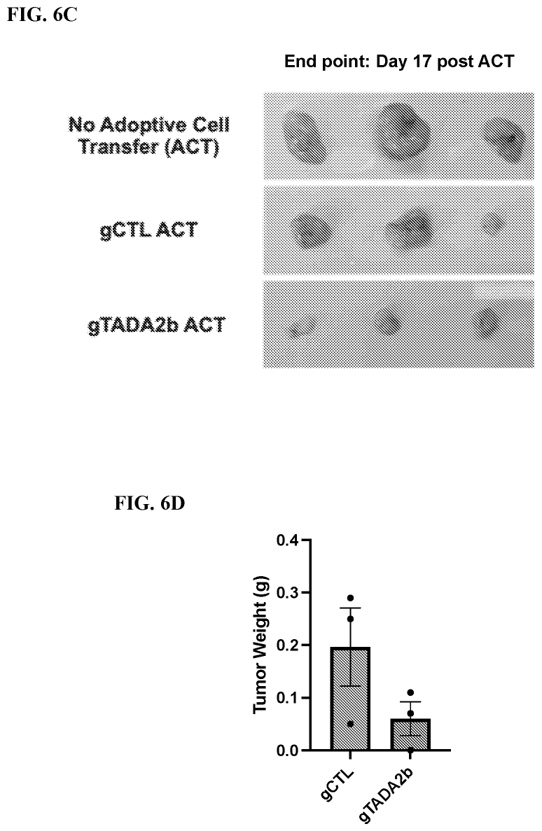

To test the ability of SAGA-depleted cells to target tumor cells in vivo, NSG mice were engrafted with 1×10⁶ A375 melanoma cells via subcutaneous injection on day 0. On day 12, 1.5×10⁶ NY-ESO-1 TCR+ T cells were infused via intravenous injection in the tail vein. A375 cell progression was measured by caliper measurements. Mice administered gTADA2b KO T NY-ESO-1 TCR+ T cells exhibited decreased tumor volume at 9 days (FIG. 6A) and 15 days (FIG. 6B), and decreased tumor weight at day 17 (FIGS. 6C-6D) post adoptive cell transfer (ACT), as compared to gC_{TL}.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific aspects have been illustrated and described, the above specification is not restrictive. It will be appreciated that various changes can be made without departing from the spirit and scope of the invention(s). Many variations will become apparent to those skilled in the art upon review of this specification.

1. A method of treating a subject in need thereof, comprising administering to the subject a population of modified immune cells, which comprises one or more modified immune cells having decreased expression of one or more components of the SAGA (Spt-Ada-Gcn5-acetyltransferase) complex, relative to an unmodified immune cell.

2. The method of claim 1, wherein the population of modified immune cells are modified by culturing the immune cells ex vivo under conditions that block or reduce the expression of one or more components of the SAGA complex prior to administration to the subject.

3. A method of treating a subject in need thereof, comprising (i) culturing a population of immune cells ex vivo under conditions that block or reduce the expression of one or more components of the SAGA thereby generating a population of modified immune cells, and (ii) administering the population of modified immune cells to the subject.

4. The method of any one of claims 1 to 3, wherein the population of immune cells comprises one or more immune cells selected from the group consisting of T cells, TSCM cells, double negative T (DNT) cells, natural killer (NK) cells, B cells, regulatory T (Treg) cells, tumor infiltrating lymphocytes, and any combination thereof.

5. The method of any one of claims 1 to 4, wherein the population of immune cells comprises CD8+ T cells.

6. The method of any one of claims 1 to 5, wherein the modified immune cell comprises a nucleic acid encoding a chimeric antigen receptor (CAR) or a nucleic acid encoding a heterologous T cell receptor (TCR).

7. The method of claim 6, wherein the CAR, the TCR, or both comprise an antigen-binding domain, wherein the antigen-binding domain specifically binds an antigen expressed on the surface of a tumor cell.

8. The method of claim 7, wherein the antigen-binding domain specifically binds an antigen selected from the group consisting of CD19, BCMA, CD30, CD33, CD123, FLT3, and any combination thereof.

9. The method of claim 7, wherein the antigen-binding domain specifically binds an antigen selected from the group consisting of NYESO-1, MAGE-A1, MAGE-A2, MAGE-A3, MAGE-A4, MAGE-A6, MAGE-A8, MAGE-A9, MAGE-A10, MAGE-A12, MART-1, gp100, WT1, tyrosinase, PRAME, p53, HPV-E6, HBV, TRAIL&DR4, thyroglobulin, TGFBII frameshift antigen, LAGE-1A, KRAS G12V, HPV-E7, HERV-E, HA-1, CMV, CEA, AFP, and any combination thereof.

10. The method of any one of claims 1 to 9, wherein the one or more components of the SAGA complex comprise USP22, TADA1, TADA2b, TAF6L, or any combination thereof.

11. The method of any one of claims 1 to 10, wherein the expression of the one or more components of the SAGA complex is inhibited or blocked by contacting the immune cells with (i) a Cas9 protein and a CRISPR-cas system guide RNA (gRNA), (ii) an antisense oligonucleotide, (iii) an siRNA, (iv) an shRNA, (v) a small molecule inhibitor, (vi) a prime editing guide RNA, or (vii) any combination thereof.

12. The method of any one of claims 1 to 11, wherein the expression of the one or more components of the SAGA complex is inhibited or blocked by contacting the immune cells with a Cas9 protein and a CRISPR-cas system guide RNA (gRNA).

13. The method of claim 11 or 12, wherein the Cas9 protein, the gRNA, or both are expressed from a heterologous expression construct introduced into the immune cell.

14. The method of any one of claims 11 to 13, wherein the gRNA is a single guide RNA (sgRNA).

15. The method of any one of claims 11 to 14, wherein the gRNA specifically hybridizes to a target DNA region within a coding region encoding a component of the SAGA complex selected from the group consisting of USP22, TADA1, TADA2b, and TAF6L.

16. The method of any one of claims 11 to 15, wherein the gRNA specifically hybridizes to a target DNA region within the USP22 coding region.

17. The method of any one of claims 11 to 15, wherein the gRNA specifically hybridizes to a target DNA region within the TADA1 coding region.

18. The method of any one of claims 11 to 15, wherein the gRNA specifically hybridizes to a target DNA region within the TADA2b coding region.

19. The method of any one of claims 11 to 15, wherein the gRNA specifically hybridizes to a target DNA region within the TAF6L coding region.

20. The method of any one of claims 11 to 19, comprising contacting the immune cell with a first gRNA and a second gRNA, wherein the first gRNA hybridizes to a target DNA region within a coding region encoding a first component of the SAGA complex, wherein the second gRNA hybridizes to a target DNA region within a coding region encoding a second component of the SAGA complex, and wherein the first component of the SAGA complex and the second component of the SAGA complex are different.

21. The method of claim 20, wherein:
(a) the first component of the SAGA complex is USP22, and the second component of the SAGA complex is TADA1;
(b) the first component of the SAGA complex is USP22, and the second component of the SAGA complex is TADA2b;
(c) the first component of the SAGA complex is USP22, and the second component of the SAGA complex is TAF6L;
(d) the first component of the SAGA complex is TADA1, and the second component of the SAGA complex is TADA2b;
(e) the first component of the SAGA complex is TADA1, and the second component of the SAGA complex is TAF6L; or
(f) the first component of the SAGA complex is TADA2b, and the second component of the SAGA complex is TAF6L.

22. The method of claim 20 or 21, further comprising contacting the immune cell with a third gRNA, wherein the third gRNA hybridizes to a target DNA region within a coding region encoding a third component of the SAGA complex; and wherein the first component of the SAGA complex, the second component of the SAGA complex, and the third component of the SAGA complex are different.

23. The method of claim 22, wherein:
(a) the first component of the SAGA complex is USP22, the second component of the SAGA complex is TADA1, and the third component of the SAGA complex is TADA2b;
(b) the first component of the SAGA complex is USP22, the second component of the SAGA complex is TADA1, and the third component of the SAGA complex is TAF6L;
(c) the first component of the SAGA complex is TADA1, the second component of the SAGA complex is TADA2b, and the third component of the SAGA complex is TAF6L; or
(d) the first component of the SAGA complex is USP22, the second component of the SAGA complex is TADA2b, and the third component of the SAGA complex is TAF6L.

24. The method of any one of claims 20 to 23, further comprising contacting the immune cell with a fourth gRNA, wherein the fourth gRNA hybridizes to a target DNA region within a coding region encoding a fourth component of the SAGA complex; and wherein the first component of the SAGA complex, the second component of the SAGA complex, the third component of the SAGA complex are different, and the fourth component of the SAGA complex are different.

25. The method of claim 24, wherein the first component of the SAGA complex is USP22, the second component of the SAGA complex is TADA1, the third component of the SAGA complex is TADA2b, and the fourth component of the SAGA complex is TAF6L.

26. The method of any one of claims 11 to 23, wherein contacting the immune cells with the Cas9 protein and the gRNA knocks out the target component of the SAGA complex in the modified immune cell.

27. The method of any one of claims 1 to 26, wherein the modified immune cells exhibit increased persistence, relative to unmodified immune cells.

28. The method of any one of claims 1 to 27, wherein the modified immune cells exhibit increased expression of one or more activation marker selected from the group consisting of CD25, HLA-DR, CD69, and any combination thereof following re-stimulation, relative to unmodified immune cells following re-stimulation.

29. The method of claim 28, wherein the re-stimulation comprises contacting the cells with CD3, CD28, CD2, or any combination thereof.

30. The method of claim 28 or 29, wherein the re-stimulation is applied at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after an initial stimulation.

31. The method of any one of claims 1 to 30, wherein the modified immune cells exhibit increased expression of one or more activation marker selected from the group consisting of CD25, HLA-DR, CD69, and any combination thereof following serial stimulation, relative to unmodified immune cells following serial stimulation.

32. The method of claim 31, wherein the serial stimulation comprises a first stimulation, a first re-stimulation, and a second re-stimulation.

33. The method of claim 32, wherein the serial stimulation further comprises a third re-stimulation.

34. The method of claim 33, wherein the serial stimulation further comprises a fourth re-stimulation.

35. The method of any one of claims 31 to 34, wherein the modified immune cells exhibit increased expression of one or more activation markers after serial stimulation, wherein the one or more activation markers are selected from the group consisting of CD25, HLA-DR, CD69, and any combination thereof at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, or at least about 41 days after an initial stimulation, relative to unmodified immune cells following serial stimulation.

36. The method of any one of claims 31 to 35, wherein the expression of the one or more activation markers is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to unmodified immune cells following re-stimulation.

37. The method of any one of claims 1 to 36, wherein the modified immune cells exhibit increased expression of CD107a following re-stimulation, relative to unmodified cells following re-stimulation.

38. The method of claim 37, wherein the expression of CD107a is increased by at least about 10%, at least about 15%, at least about 20%, at least about 25%, at least about 30%, at least about 40%, at least about 50%, at least about 75%, or by at least about 100%, relative to unmodified cells following re-stimulation.

39. The method of any one of claims 1 to 38, wherein the modified immune cells exhibit decreased expression of one or more T cell exhaustion markers following re-stimulation, relative to unmodified immune cells.

40. The method of claim 39, wherein the one or more T cell exhaustion markers are selected from the group consisting of LAG3 and TIM3.

41. The method of claim 39 or 40, wherein the expression of the one or more T cell exhaustion markers is decreased by at least about 10%, at least about 15%, at least about 20%, at least about 25%, at least about 30%, at least about 40%, at least about 50%, or at least about 75%, relative to unmodified cells following re-stimulation.

42. The method of any one of claims 1 to 41, wherein the subject is afflicted with a cancer.

43. The method of claim 42, wherein the cancer is selected from the group consisting of melanoma, bone cancer, renal cancer, prostate cancer, breast cancer, colon cancer, lung cancer, cutaneous or intraocular malignant melanoma, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of the cancers.

44. The method of claim 42 or 43, wherein the cancer is relapsed, refractory, advanced, and/or metastatic.

45. The method of any one of claims 1 to 44, further comprising administering to the subject an additional anticancer agent.

46. The method of claim 45, wherein the additional anticancer agent comprises an immunotherapy, a chemotherapy, a cytokine, a radiation therapy, a surgery, or any combination thereof.

47. The method of claim 45 or 46, wherein the additional anticancer agent comprises a cell based immunotherapy, an antibody or an antigen-binding portion thereof, or both.

48. The method of any one of claims 45 to 47, wherein the additional anticancer agent comprises a checkpoint inhibitor.

49. The method of any one of claims 45 to 48, wherein the additional anticancer agent comprises a PD-1 inhibitor, a PD-L1 inhibitor, a CTLA-4 inhibitor, a LAG3 inhibitor, a TIM3 inhibitor, or any combination thereof.

50. The method of any one of claims 45 to 49, wherein the additional anticancer agent comprises an antibody or an antigen-binding portion thereof that specifically binds PD-1 or PD-L1.

51. The method of any one of claims 1 to 50, wherein the modified immune cells exhibit increased phospho-MEK levels following stimulation, relative to unmodified cells following stimulation.

52. The method of any one of claims 1 to 51, wherein the modified immune cells exhibit increased phospho-ATK levels following stimulation, relative to unmodified cells following stimulation.
